(19) United States
(12) Patent Application Publication
(10) Pub. No.: US 2014/0112602 A1
(43) Pub. Date: Apr. 24, 2014

(54) SYNERGISTICALLY-EFFECTIVE COMPOSITION OF ZINC RICINOLEATE AND SUBSTITUTED MONOCYCLIC ORGANIC COMPOUNDS AND USE THEREOF FOR REDUCING MALODORS

(71) Applicants: Stephen P. Nicoll, Summit, NJ (US); Krystyna Malgorzata Rankin, Ridgewood, NJ (US)

(72) Inventors: Stephen P. Nicoll, Summit, NJ (US); Krystyna Malgorzata Rankin, Ridgewood, NJ (US)

(21) Appl. No.: 13/657,918

(22) Filed: Oct. 23, 2012

(51) Int. Cl. A61L 9/01; B65F 1/00

(52) U.S. Cl. 383/105; 424/76.1

(57) ABSTRACT

Described is a novel zinc ricinoleate-containing malodor reducing composition further comprising:
(a) cyclohexanol, 4-isopropyl: propionate, cis;
(b) cyclohexanemethanol, alpha-methyl: acetate;
(c) butyric acid: 1-cyclohexylethyl ester; and
(d) cyclohexanemethanol, alpha-methyl,
wherein the ratio of (a):(b):(c):(d) is about 2.8-4.8:0.5-2.5:0.1-1:0.25-1, and wherein zinc ricinoleate is at a level of about 1-3% by weight. Also described for the purpose of application to an inanimate laminar substantially solid surface are malodor-suppressing composition-containing stick articles containing an ester-terminated polyamide or an amide-terminated polyamide structural support polymer in combination with the aforementioned zinc ricinoleate-containing malodor reducing compositions. Also described is a package for conveniently handling the aforementioned stick article.

### STATUS OF RELATED APPLICATIONS

This application is a continuation-in-part of U.S. Ser. No. 12/343,129, filed Dec. 23, 2008, now pending, which is a continuation-in-part of U.S. Ser. No. 10/706,888, filed Nov. 13, 2003, now abandoned, the contents hereby incorporated by references as if set forth in their entirety.

### FIELD OF THE INVENTION

A novel zinc ricinoleate-containing malodor reducing composition further comprising:
(a) cyclohexanol, 4-isopropyl: propionate, cis;
(b) cyclohexanemethanol, alpha-methyl: acetate;
(c) butyric acid: 1-cyclohexylethyl ester; and
(d) cyclohexanemethanol, alpha-methyl,
wherein the ratio of (a):(b):(c):(d) is about 2.8-4.8:0.5-2.5:0.1-1:0.25-1, and wherein zinc ricinoleate is at a level of about 1-3% by weight.

### BACKGROUND OF THE INVENTION

A wide variety of solid, liquid and gaseous functional materials including body deodorants, anti-perspirants, anti-perspirant/body deodorant devices, air fresheners which include air freshening devices, and solid and liquid air freshening and room freshening compositions, room deodorants, herbicides, antiviral compositions, fungicides, bactericides, parasiticides, insecticides, depilatory compositions, bleach compositions, hard surface-cleaning compositions, skin cleansing compositions, anti-microbial nail preparations including anti-fungal nail lacquers, hair setting compositions, hair conditioning compositions, trichological lotions, detergent compositions, soap compositions, sunscreen compositions, fabric stain-removal compositions, fabric softener compositions, fabric conditioning compositions, fabric anti-wrinkle compositions, skin softening compositions, skin texture enhancement compositions, such as line and wrinkle corrective compositions, skin lightening compositions, steam iron aroma compositions including stress relief compositions, candle compositions, plant growth regulating compositions, plant growth stimulating compositions, fertilizer compositions, insect attractant compositions, insect repelling compositions, drain cleaning compositions and molluskicide compositions have been developed that, although useful for their respective purposes, on use thereof emanate odors which are offensive to the human sense of smell.

In addition, spaces which are used for various business and service operations and personal matters including indoor gymnasiums, indoor sporting event arenas, locker rooms, hair salons, nail salons, tanning salons, beauty salons, tattoo parlors, pig pens, chicken coops, cow barn enclosures, horse barn enclosures, indoor fresh fish markets, plant processing factory rooms, clothing dry cleaning rooms, garment laundry interiors, rooms containing in-use animal litter containers, abattoirs, cattle cars, zoo animal pens, morgues, autopsy rooms, lavatories, medical patient care rooms, hospital wards and dental patient care rooms have continuously prevailing odors which are offensive to the human sense of smell. Such odors which are offensive to the human sense of smell are caused by aliphatic halohydrins, aliphatic amines, aliphatic N-oxides, dialkylamines, cycloaliphatic amines, cycloaliphatic N-oxides, cyclo-olefinic amines, cyclo-olefinic N-oxides, cycloaromatic amines, cycloaromatic N-oxides, hydroxyalkylamines, imine compounds, amide compounds, amino acids, polypeptides, modified antimicrobial proteins, diureides, nitriles, aliphatic mercaptans, cycloaliphatic mercaptans, mercaptoalkanoic acids, mercaptoalkanoic acid esters, aliphatic monosulfides, disulfides, trisulfides, sulfur oxides, sulfones and sultones, cycloaliphatic monosulfides, disulfides, trisulfides, sulfur oxides, sulfones and sultones, cyclo-olefinic monosulfides, disulfides, trisulfides, sulfur oxides, sulfones and sultones, cycloaromatic monosulfides, disulfides, trisulfides, sulfur oxides, sulfones and sultones, alkali metal sulfites, bisulfites and metabisulfites, isothiocyanates, thiocyanates, dithiocyanates, isothiazolones, isothiazolinones, thiodiazinethiones, halosulfamates, aryl sulfonamides, lower aliphatic carboxylic acids, phenols, phosphines, aliphatic phosphites and phosphonates, cycloaliphatic phosphites and phosphonates, arsines, lower alcohols, lower ketones, hops, hops acids, aryl pyrazoles, oxazolines, isocyanurates, biguanides, extracts of krameria, hydantoins, pyrollidones, pyrollidone carboxylic acids, pyrollidone carboxylic acid esters, nitrophenols, N-substituted aspartic acids and pyrethroids. Compounds of these classes that have unpleasant odors are referred to herein as malodor compounds.

The aforementioned functional materials are known to include and/or have applied thereto or in a space proximate thereto, fragrance materials that are intended to provide pleasant fragrances which mask the malodor. In addition, the aforementioned spaces are known to have introduced therein compositions such as fragrance materials that are intended to provide pleasant aromas which mask the malodor, present therein and/or introduced thereto.

The masking effect is provided by one of three mechanisms. In the first mechanism, the masking composition components blend with the malodor compound or compounds in an effort to effect formation of a different and more desirable aroma. In the second mechanism the masking composition components are employed in a large quantity and concentration whereby the compound or composition responsible for the malodor is overwhelmed. In the third mechanism, one or more masking composition components overwhelm the compound or composition responsible for the malodor and one or more masking composition components chemically react with the malodor compound or composition thereby effecting formation of a more desirable aroma.

Each of the aforementioned mechanisms has serious disadvantages. Thus, in the case where a fragrance composition is used as a malodor maskant, it will not completely eliminate the perception of malodor and, accordingly, there is a tendency to use increasing quantities and concentrations of fragrance in an effort to eliminate the perception of malodor. Furthermore, the masking effect is an additive effect and so the total odor level in the malodor-masked functional product or in the malodor-masked space is increased by consumption of the fragrance. Even though the fragrance so used may be very pleasant at low concentrations, the total odor level in the space proximate the in-use functional product and the total odor level in the aforementioned space at the relatively high concentrations required to achieve even moderate masking of the malodor will itself be offensive to the human sense of smell.

The prior art contains evidence of a large number of efforts to overcome the aforementioned disadvantages. Thus, the use of zinc ricinoleate, also known as TEGO Sorb (Goldschmidt A.G. of Essen, Germany) for suppressing malodors is disclosed in U.S. Pat. No. 6,528,047, U.S. Pat. No. 6,592,813, U.S. Pat. No. 4,968,496 and U.S. Patent Application 2003/007945 A1 published on Jan. 9, 2003. Furthermore the, use of one or more of the substituted cyclic organic compounds:
cyclohexanol, 4-isopropyl: propionate, cis;
cyclohexanemethanol, alpha-methyl: acetate;
butyric acid: 1-cyclohexylethyl ester; and
cyclohexanemethanol, alpha-methyl,
   sold under Veilex (International Flavors & Fragrances Inc., New York, N.Y.) for suppressing malodors are disclosed in, for example, in U.S. Pat. No. 6,432,891, U.S. Pat. No. 6,592,813 and U.S. Pat. No. 4,622,221.

Furthermore, the use of zinc ricinoleate in functional substance such as fragrance containing polyamide sticks and packages dispensing same are disclosed in U.S. patent application Ser. No. 10/683,525 filed on Oct. 10, 2003, but no mention is made therein of the use of polyamide sticks for malodor suppression or prevention.

However, nothing in the prior art discloses the novel zinc ricinoleate-containing malodor reducing composition of the present invention, which further comprises:
(a) cyclohexanol, 4-isopropyl: propionate, cis;
(b) cyclohexanemethanol, alpha-methyl: acetate;
(c) butyric acid: 1-cyclohexylethyl ester; and
(d) cyclohexanemethanol, alpha-methyl,

wherein the ratio of (a):(b):(c):(d) is about 2.8-4.8:0.5-2.5:0.1-1:0.25-1, and wherein zinc ricinoleate is at a level of about 1-3% by weight.

### SUMMARY OF THE INVENTION

The present invention is directed to a novel zinc ricinoleate-containing malodor reducing composition further comprising:

(a) cyclohexanol, 4-isopropyl: propionate, cis (Veilex No. 4) having the structure:

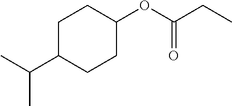

(b) cyclohexanemethanol, alpha-methyl: acetate (Veilex No. 2) having the structure:

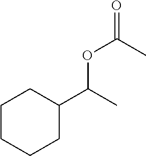

(c) butyric acid: 1-cyclohexylethyl ester (Veilex No. 1) having the structure:

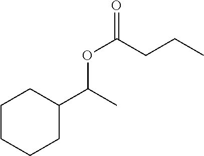

and (d) cyclohexanemethanol, alpha-methyl (Veilex No. 3) having the structure:

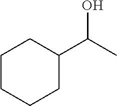

wherein the ratio of (a):(b):(c):(d) is about 2.8-4.8:0.5-2.5:0.1-1:0.25-1, and wherein zinc ricinoleate is at a level of about 1-3% by weight.

Zinc ricinoleate is selected from the group consisting of zinc ricinoleate and solutions of zinc rincinoleate containing greater than 30% by weight of zinc ricinoleate. Preferably, the zinc ricinoleate of the present invention is a mixture of about 50% by weight of zinc ricinoleate and about 50% by weight of at least one 1-hydroxy-2-ethoxethyl ether of a C₁₂-C₁₄ fatty alcohol.

The present invention is also directed to a process for counteracting a malodor emanating from a solid or liquid malodorous source into a space proximate said source (e.g., malodorous garbage contained in a polyethylene-fabricated garbage bag) comprising the step of introducing into the space proximate said source the zinc ricinoleate-containing malodor reducing composition of the present invention as a single dose, as a continuous dose over a malodor-counteracting period of time, or as periodic doses over a malodor-counteracting period of time whereby the perceived total malodor intensity is substantially reduced or eliminated.

The present invention is also directed to a process for preventing a malodor from emanating from a solid or liquid malodorous source into a space proximate said source comprising the step of admixing with said source the zinc ricinoleate-containing malodor reducing composition of the present invention.

More specifically, the present invention is directed to a process wherein the solid or liquid malodorous source evolving or capable of evolving the malodor is, for example, one of: a herbicide; an antiviral composition; a fungicide; a bactericide; a parasiticide; an insecticide; a depilatory preparation; a bleach composition; a hard surface-cleaning preparation; a skin cleansing composition; an anti-microbial nail preparation; a hair setting composition; a hair conditioning composition; a trichological lotion; a skin lightening composition; a skin softening composition; a skin texture enhancement composition; a detergent composition; a soap composition; a sunscreen composition; a fabric stain removal composition; a fabric softening composition; a fabric conditioning composition; a fabric anti-wrinkle composition; a steam iron aroma composition; a candle composition; a plant growth regulating composition; a plant growth stimulating composition; a fertilizer composition; an insect attractant composition; an insect repelling composition; a drain cleaning composition; a molluskicide composition; an antiperspirant composition; a body deodorant composition; a body deodorant/anti-perspirant device; a waste-disposal container; an air freshener device and an air freshener composition; and wherein the malodor is caused by, for example, a malodor-causing quantity and concentration of at least one compound selected from the group consisting of malodor compounds as described herein.

In addition, in order to effect delivery of the zinc ricinoleate-containing malodor reducing composition of the present invention to an inanimate laminar surface such as the surface of a polypropylene garbage bag containing malodor-evolving garbage, another aspect of the present invention is directed to a malodor-suppressing stick article having a consistently-maintained system-compatible malodor-suppressing composition of the present invention and dimensional integrity comprising a stiff, substantially monophasic, thermally-reversible composition comprising:

(a) a structural support polymer which is, in the alternative or in combination an ester-terminated polyamide (herein also referred to as ETPA) and/or a tertiary amide-terminated polyamide (herein also referred to as ATPA); and

(b) a composition of zinc ricinoleate and substituted monocyclic organic compounds as described supra,

with the provisos that the weight ratio range of the structural support polymer and the zinc ricinoleate-containing malodor reducing composition of the present invention is from about 90:10 to about 55:45 and the weight percent of zinc ricinoleate in the stick article is from about 0.5% to about 10% by weight of the stick article. The stick article of the present invention may optionally also contain other system compatible functional compositions including, but not limited to insect repellents, anti-microbial compositions, fragrance components, air freshener compositions and animal repellent compositions.

In one embodiment of the invention, a malodor reducing garbage bag system comprising, in combination, a garbage bag including an impermeable, flexible inelastic sheet constructed from a polyethylene material and including a side wall with a closed bottom and an open top; a malodor reducing composition impregnated within the sheet of the garbage bag.

In a further embodiment of the present invention, the malodor reducing garbage bag system contains a malodor reducing composition comprising the following ingredients:
cyclohexanol, 4-isopropyl: propionate, cis;
cyclohexanemethanol, alpha-methyl: acetate;
butyric acid: 1-cyclohexylethyl ester;
cyclohexanemethanol, alpha-methyl;
zinc ricinoleate;
and mixtures thereof.

In another further embodiment, the malodor reducing composition comprises:
about 2.8-4.8% by weight of cyclohexanol, 4-isopropyl: propionate, cis;
about 0.5-2.5% by weight of cyclohexanemethanol, alpha-methyl: acetate;
about 0.1-1% by weight of butyric acid: 1-cyclohexylethyl ester;
about 0.25-1% by weight of cyclohexanemethanol, alpha-methyl; and
about 1-3% by weight of zinc ricinoleate.

In another further embodiment, the malodor reducing composition comprises:
about 3.8% by weight of cyclohexanol, 4-isopropyl: propionate, cis;
about 1.5% by weight of cyclohexanemethanol, alpha-methyl: acetate;
about 0.4% by weight of butyric acid: 1-cyclohexylethyl ester;
about 0.5% by weight of cyclohexanemethanol, alpha-methyl; and
about 2% by weight of zinc ricinoleate.

In another further embodiment, the malodor reducing composition is a zinc ricinoleate-containing composition further comprising:
(a) cyclohexanol, 4-isopropyl: propionate, cis;
(b) cyclohexanemethanol, alpha-methyl: acetate;
(c) butyric acid: 1-cyclohexylethyl ester; and
(d) cyclohexanemethanol, alpha-methyl,

wherein the ratio of (a):(b):(c):(d) is about 2.8-4.8:0.5-2.5:0.1-1:0.25-1, and wherein zinc ricinoleate is at a level of about 1-3% by weight.

In another further embodiment, the malodor reducing composition is a zinc ricinoleate-containing composition further comprising:
(a) cyclohexanol, 4-isopropyl: propionate, cis;
(b) cyclohexanemethanol, alpha-methyl: acetate;
(c) butyric acid: 1-cyclohexylethyl ester; and
(d) cyclohexanemethanol, alpha-methyl,

wherein the ratio of (a):(b):(c):(d) is about 3.8:1.5:0.4:0.5, and wherein zinc ricinoleate is at a level of about 2% by weight.

In another further embodiment, the malodor reducing composition additionally comprises a solvent.

In another further embodiment, a malodor reducing garbage bag system contains about 1-5% by weight of the malodor reducing composition of the present invention.

In another further embodiment, a substantially fragrance free odor blocking garbage bag system comprising, in combination, a garbage bag including an impermeable, flexible inelastic sheet constructed from a polyethylene material and including a side wall with a closed bottom and an open top; and the malodor reducing composition of the present invention impregnated within the sheet of the garbage bag.

The terms system-compatible malodor-suppressing composition and system-compatible functional composition are herein intended to mean the malodor-suppressing , compositions of the present invention and other functional compositions, for example, fragrance compositions which, when made part of the support polymer-zinc ricinoleate/substituted monocyclic organic compounds system do not compromise the transparency of the stick article by causing haze or cloudiness, due to, for example, phase separation or synerisis to occur as a result of the composition being admixed with the support polymer-zinc ricinoleate/substituted monocyclic organic compounds system of the present invention. The term consistently-maintained malodor-suppressing composition and dimensional integrity is intended herein to mean that when the stick article of the present invention is in use in applying a film to an inanimate solid laminar surface, the proportions of the constituents and the chemical properties of the malodor-suppressing composition of the present invention and, optionally, other functional composition, such as a the present invention are substantially identical to the proportions and chemical properties of the constituents originally present in the stick article and originally admixed with the support polymer-zinc ricinoleate/substituted monocyclic organic compounds system.

The term stiff is herein intended to mean that the stick article of the present invention is self-supporting and non-flowable at ambient temperatures or less and at ambient pressures, e.g., at temperatures of less than or equal to 35° C. and at pressures of about 1 atmosphere absolute.

The term monophasic is herein intended to mean that the stick article of the present invention on use or when not in use exists in one unitary phase without any phase separation resulting from the inclusion in the support polymer-zinc ricinoleate/substituted monocyclic organic compounds system of a functional composition, e.g., a malodor-suppressing or preventing composition or an optional fragrance composition.

The term thermally reversible is herein intended to mean that the stick article of the present invention retains the original proportions of the constituents of its composition and retains its original physical characteristics and original dimensions on use thereof, and subsequent to use thereof.

The term malodor reducing is herein intended to mean the ability to reduce the perceived malodor intensity, for example, in a garbage bag system.

The term odor-blocking is herein intended to mean the reduction of perceived malodor intensity, for example, in a garbage bag system.

The term substantially fragrance free is herein intended to mean wherein fragrance is not detectable in the garbage bag system.

The present invention is also directed to packages containing such stick articles, and a process for employing such stick articles whereby an inanimate laminar surface is controllably coated with a thin film of ETPA or ATPA polymer which (a) effects malodor suppression at and proximate to said inanimate laminar surface and (b) releases the volatile components of the malodor-suppressing composition at a controllable rate into the environment proximate said inanimate laminar surface and (c) optionally releases aesthetically pleasing fragrance at a controllable rate into the environment proximate said inanimate laminar surface. The coating is effected, for example, by means of manually rubbing an exposed end of the malodor-suppressing stick of the present invention onto the said inanimate laminar solid surface, using a package containing a partially-exposed malodor-suppressing stick article of the present invention.

### DETAILED DESCRIPTION OF THE INVENTION

The zinc ricinoleate-containing malodor reducing composition of the present invention is preferably utilized in combination with a solvent which is at least one ethoxylated C₁₂-C₁₄ fatty alcohol, preferably one or more of a 1-hydroxy-2-ethoxyethyl ether of a C₁₂-C₁₄ fatty alcohol, for example, the 1-hydroxy-2-ethoxyethyl ether of stearyl alcohol in a concentration of greater than 30%, preferably at a concentration of about 50%.

The zinc ricinoleate-containing malodor reducing composition of the present invention further comprises:
(a) cyclohexanol, 4-isopropyl: propionate, cis;
(b) cyclohexanemethanol, alpha-methyl: acetate;
(c) butyric acid: 1-cyclohexylethyl ester; and
(d) cyclohexanemethanol, alpha-methyl,

wherein the ratio of (a):(b):(c):(d) is about 2.8-4.8:0.5-2.5:0.1-1:0.25-1, and wherein zinc ricinoleate is at a level of about 1-3% by weight.

More preferably, the zinc ricinoleate-containing malodor reducing composition recited in the above, wherein the ratio of (a):(b):(c):(d) is about 3.8:1.5:0.4:0.5, and wherein zinc ricinoleate is at a level of about 2% by weight.

As stated above, the zinc ricinoleate-containing malodor reducing composition of the present invention substantially eliminates the perception of malodors, and/or prevents the formation of such malodors while simultaneously refraining from reduction of the perception of pleasant fragrance aromas emanating from the same source or from the proximity of said source. The zinc ricinoleate-containing malodor reducing composition of the present invention can be utilized with a vast number of functional products in order to prevent malodors evolved by such functional products during use thereof and/or to suppress malodors evolved by such functional products into the space proximate the functional products while they are being used.

Examples of such functional products which are compositions, effective specific malodorous ingredients or classes of ingredients contained in and/or emanated from said functional products and exemplary defined air dimensional spaces where such functional products are used, together with U.S. patent references setting forth specific examples of the utilities of such functional products are set forth in the following Table I:

TABLE I

| Functional | U.S. Pat. | | |
|---|---|---|---|
| Product | No. | Ingredient or Ingredient Class | Air Space |
| hair fixative | 5,968,494 | 2,2-hydroxymethyl-substituted | hair salons |
| composition | | carboxylic acid | |
| hair fixative | 6,291,580 | polyurethanes and diisocyanates | hair salons |
| anti-microbial | 6,294,186 | retinoids | health care facilities |
| compositions | | | |
| pesticides | 6,488,949 | thiacloprid | plant greenhouse |
| laundry and cleaning | 6,489,279 | xyloglucanase enzymes | laundries |
| compositions | | | |
| fungicides | 6,489,348 | substituted benzopyridyl ethers | hospital ward |
| fungicides | 6,489,360 | phenyl benzyl ether imines, | plant greenhouse |
| | | carbamates and dinitrophenols | |
| antibacterial liquid | 6,492,313 | amine oxides and sodium paraffin | kitchens |
| detergents | | sulfonates | |
| cleaning wipes | 6,492,318 | amine oxides | kitchens |
| floor | 6,492,499 | cocoamido-propyldimethyl amine | kitchens |
| cleaning wipes | | oxide | |
| skin treatment | 6,531,142 | sulfopolyester resins | health care facilities |
| compositions | | | |
| fabric care products | 6,531,444 | polyalkyleneimines | clothing dry cleaning |
| | | | rooms |
| cleaning compositions | 6,465,404 | coconut diethanolamide | laundries |
| dish cleaning | 6,465,406 | ethylenediamine-N,N-disuccinate | kitchens |
| compositions | | | |
| laundry detergents | 6,465,410 | N-vinylimidazole N-vinyl | laundries |
| | | pyrrolidone copolymers | |
| pesticides | 6,468,555 | fluorophenyl oxazolines | plant greenhouses |
| antimicrobial | 6,471,974 | N-chlorosulfamates | health care facilities |
| compositions | | | |
| liquid | 6,472,361 | sodium cumene sulfonate | kitchens |
| cleaning compositions | | | |
| biocide compositions | 6,475,505 | isothiazolinones | health care facilities |
| anti-viral | 6,475,526 | t-butyl alkoxyphenols | health care facilities |
| compositions | | | |
| antimicrobial | 6,475,536 | extract of krameria | beauty salons |
| cosmetic formulations | | | |
| antimicrobial | 6,475,537 | hops acids | health care facilities |
| compositions | | | |
| cleaning compositions | 6,479,449 | triethanolamine | kitchens |
| aerosol antimicrobial | 6,482,392 | amine oxides | kitchens |
| compositions | | | |
| antimicrobial wipes | 6,482,423 | 2-pyrrolidone-5-carboxylic acid | kitchens |
| parasiticidal | 6,482,425 | imidazolyl pyridines | indoor farm facilities |
| compositions | | | |
| antimicrobial wipes | 6,488,943 | halogenated carbanilides | kitchens |
| antibacterial | 6,488,948 | behentrimonium methosulfate | beauty salons |
| compositions for skin | | | |
| care | | | |
| nail care compositions | 6,503,488 | hydrogenated castor oil | beauty salons |
| cosmetic and skin | 6,613,338 | benzimidazole sulfonic acids | beauty salons |
| care sticks | | | |

Preferably, the zinc ricinoleate-containing malodor reducing composition is employed in compatible solvents prior to the composition being incorporated into functional products. Such compatible solvents are, preferably, 1,2-propylene glycol, and di(1,2-dihydroxypropyl)ether, commonly known as dipropylene glycol or DPG. The range of dilution of the zinc ricinoleate-containing malodor reducing composition in the compatible solvents is from about 2% to about 50% by weight, preferably from about 10% to about 30%, and more preferably about 15% by weight.

When used in conjunction with malodorous solid or liquid functional products, e.g., soap and detergent compositions, the zinc ricinoleate-containing malodor reducing composition of the present invention may be employed in a concentration of from about 75 to about 300 ppm based on the weight of the solid or liquid composition, preferably from about 100 to about 250 ppm.

When used in conjunction with malodorous gaseous functional products, the zinc ricinoleate-containing malodor reducing composition of the present invention may comprise from about 1.0 to about 5.0 grams/liter, preferably from about 1.5 to about 3.0 grams/liter of the air space which contains the malodor to be counteracted.

As stated above, the functional products with which the zinc ricinoleate-containing malodor reducing composition of the present invention may be used also may optionally include fragrances, anti-microbial substances, animal repellents, insect attractants, and insect repellents, each of the components of which has a C log₁₀P in the range of from about 3.0 to about 8.0, without restriction on the molecular weight of each of said components wherein P is the n-octanol/water partition coefficient of the fragrance component. Examples of fragrance/anti-microbial compositions useful in the practice of the present invention are set forth in U.S. Pat. No. 6,495,512 and U.S. Pat. No. 6,517,759.

The values of Clog₁₀P of many functional product ingredients, for example, fragrance ingredients and/or antimicrobial ingredients, contained in personal treatment compositions and/or cosmetic compositions is discussed in U.S. Patent Application US 2003/0110682 A1 published on Jun. 19, 2003, U.S. Pat. No. 5,348,667, U.S. Pat. No. 5,652,206 and U.S. Pat. No. 6,528,013. Furthermore, values of log₁₀P have been reported; for example, the Pomona92 database, available from Daylight Chemical Information Systems, Inc., Daylight CIS, Irvine, Calif. However, the log₁₀P values are most conveniently calculated by the “CLOGP” program, also available from Daylight CIS. This program also lists experimental log₁₀P values when they are available in the Pomona92 database. The calculated log₁₀P (Clog₁₀P) is determined by the Hansch and Leo “fragment” approach based on the chemical structure of each perfume ingredient, and takes into account the numbers and types of atoms, the atom connectivity and the chemical bonding. The Clog₁₀P values which are the most reliable and widely used estimates for this physicochemical property, are preferably used instead of the experimental log₁₀P values for the selection of functional ingredients, including perfume ingredients which are useful in the stick articles of the present invention.

Specific examples of preferred fragrance components useful in the stick articles of the present invention, and the molecular weights and Clog₁₀P values of each of said components are set forth in Table II as follows:

TABLE II

| Fragance Component | Clog₁₀P value | Molecular Weight |
|---|---|---|
| amyl salicylate | 4.601 | 208.26 |
| benzyl salicylate | 4.383 | 228.25 |
| β-caryophyllene | 6.333 | 204.36 |
| cedrol | 4.530 | 222.37 |
| ethyl undecylenate | 4.888 | 212.34 |
| geranyl anthranilate | 4.216 | 273.38 |
| α-irone | 3.820 | 206.33 |
| phenyl ethyl benzoate | 4.058 | 226.28 |
| phenylethyl phenyl acetate | 3.767 | 240.31 |
| 1-phenyl hexanol-5 | 3.299 | 178.28 |
| α-santalol | 3.800 | 220.36 |
| amyl salicylate | 4.601 | 208.26 |
| benzyl salicylate | 4.383 | 228.25 |
| β-caryophyllene | 6.333 | 204.36 |
| cedrol | 4.530 | 222.37 |
| cedryl acetate | 5.436 | 264.41 |
| cedryl formate | 5.070 | 238.37 |
| cyclohexyl salicylate | 5.265 | 220.29 |
| γ-dodecalactone | 4.359 | 198.31 |
| ethyl undecylenate | 4.888 | 212.34 |
| geranyl anthranilate | 4.216 | 273.38 |
| β-phenylethyl benzoate | 4.058 | 226.38 |
| β-phenylethyl phenyl acetate | 3.767 | 240.31 |
| 5-acetyl-1,1,2,3,3,6-hexamethyl | 5.977 | 258.41 |
| cyclopentadecanolide | 6.246 | 240.39 |
| d-limonene | 4.232 | 136.24 |
| cis-p-t-butylcyclohexyl acetate | 4.019 | 198.31 |
| amyl cinnamic aldehyde | 4.324 | 202.30 |
| linalyl benzoate | 5.233 | 258.36 |

As stated above, the zinc ricinoleate-containing malodor reducing composition of the present invention may be employed in stick articles having an ETPA or ATPA polymeric structure. Thus, the support polymer used in the stick article of the present invention is, in the alternative or in combination (a) at least one ester-terminated polyamide or (b) at least one tertiary amide-terminated polyamide.

Preferable ester-terminated polyamides useful in the practice of the present invention are those disclosed in U.S. Pat. No. 5,783,657 and U.S. Pat. No. 6,552,160 and include those ester-terminated polyamides prepared by reacting “x” equivalents of a dicarboxylic acid wherein at least 50% of those equivalents are from polymerized fatty acid, “y” equivalents of ethylenediamine and “z” equivalents of an alcohol which is in the alternative, or in combination cetyl alcohol and/or stearyl alcohol wherein:

$$0.9 \leq \frac{X}{y + z} \leq 1.1 and 0.1 \leq \frac{z}{y + z} \leq 0.7$$

More preferably, the ester-terminated polyamide is one of a group having a weight-average molecular weight of about 6000 and a softening point in the range of from 88°°C. to 94° C. prepared by reacting “x” equivalents of C₃₆ dicarboxylic acid, “y′ equivalents of ethylenediamine and “z′ equivalents of an alcohol which is, in the alternative or in combination cetyl alcohol and/or stearyl alcohol wherein

$$0.09 \leq \frac{X}{y + z} \leq 1.1 and 0.1 \leq \frac{z}{y + z} \leq 0.7 .$$

Most preferable are the mineral oil-free ester terminated polyamides, SYLVACLEAR 100LM, UNICLEAR 100 and UNICLEAR 100V, Arizona Chemical Company, Panama City, Fla.

Preferable tertiary amide-terminated polyamides useful in the practice of the present invention are those disclosed in U.S. Pat. No. 6,268,466 and U.S. Pat. No. 6,469,131 and include those tertiary amide-terminated polyamides prepared by reacting “x” equivalents of dicarboxylic acid wherein at least 50% of those equivalents are from polymerized fatty acid, “y” equivalents of ethylenediamine and “z” equivalents of a monofunctional reactant having a secondary amine group as the only reactive functionality wherein

$$0.09 \leq \frac{X}{y + z} \leq 1.1 and 0.1 \leq \frac{z}{y + z} \leq 0.7$$

Most preferable are those tertiary amide-terminated polyamides disclosed in Example I of U.S. Pat. No. 6,268,466.

The stick articles of the present invention, when used in a preferred manner in the practice of the present invention are contained, for example, in a package as illustrated in FIGS. 1, 2 and 3 described infra. Packages which are so useful in the practice of the present invention are described in detail in U.S. Pat. Nos. 5,275,496 and 6,386,778 and U.S. Patent Application US 2001/0002962 A1 published on Jun. 7, 2001.

Such packages for the stick article of the present invention comprise means for supporting the functional substance-providing stick article of the present invention such that an end portion of said stick article can be exposed for use, with the means for supporting said stick article including a stick-surrounding member for surrounding the stick article, the stick-surrounding member having an opening such that the stick article can be exposed for use, and a border of the stick-surrounding member forming the opening, said border forming a top end of the supporting means, such that said end portion can be elevated from said means for supporting and can protrude therefrom so as to be exposed for use, said stick article being contained within said stick-surrounding member. Thus for example, the means for elevating includes a screw feed mechanism having an elevator screw such that upon rotation of the elevator screw the stick article is pushed up from the bottom such that the end portion protrudes and is exposed for use. As a second example, the means for elevating is a means for pushing up the stick article so that the end portion protrudes from the means for supporting and is exposed for use.

Furthermore, as stated above, the present invention is directed to a process for applying a controllably releasable functional substance such as a perfume composition, an insect repellant composition, an animal repellant composition, an anti-microbial composition, an insect attractant composition and/or an air freshener composition to an inanimate laminar substantially solid surface comprising the steps of providing a package as described supra; exposing for use the end portion of said stick article contained within said package; providing an inanimate laminar substantially solid surface to be treated; and applying the end portion of said stick article contained within said package to a finite area of said laminar substantially solid surface.

In one embodiment of the invention, a malodor reducing garbage bag system comprising, in combination, a garbage bag including an impermeable, flexible inelastic sheet constructed from a polyethylene material and including a side wall with a closed bottom and an open top; a malodor reducing composition impregnated within the sheet of the garbage bag.

In a further embodiment of the present invention, the malodor reducing garbage bag system contains a malodor reducing composition comprising the following ingredients:
cyclohexanol, 4-isopropyl: propionate, cis;
cyclohexanemethanol, alpha-methyl: acetate;
butyric acid: 1-cyclohexylethyl ester;
cyclohexanemethanol, alpha-methyl;
zinc ricinoleate;
and mixtures thereof.

In another further embodiment, the malodor reducing composition comprises:
about 2.8-4.8% by weight of cyclohexanol, 4-isopropyl: propionate, cis;
about 0.5-2.5% by weight of cyclohexanemethanol, alpha-methyl: acetate;
about 0.1-1% by weight of butyric acid: 1-cyclohexylethyl ester;
about 0.25-1% by weight of cyclohexanemethanol, alpha-methyl; and
about 1-3% by weight of zinc ricinoleate.

In another further embodiment, the malodor reducing composition comprises:
about 3.8% by weight of cyclohexanol, 4-isopropyl: propionate, cis;
about 1.5% by weight of cyclohexanemethanol, alpha-methyl: acetate;
about 0.4% by weight of butyric acid: 1-cyclohexylethyl ester;
about 0.5% by weight of cyclohexanemethanol, alpha-methyl; and
about 2% by weight of zinc ricinoleate.

In another further embodiment, the malodor reducing composition is a zinc ricinoleate-containing composition further comprising:
(a) cyclohexanol, 4-isopropyl: propionate, cis;
(b) cyclohexanemethanol, alpha-methyl: acetate;
(c) butyric acid: 1-cyclohexylethyl ester; and
(d) cyclohexanemethanol, alpha-methyl,

wherein the ratio of (a):(b):(c):(d) is about 2.8-4.8:0.5-2.5:0.1-1:0.25-1, and wherein zinc ricinoleate is at a level of about 1-3% by weight.

In another further embodiment, the malodor reducing composition is a zinc ricinoleate-containing composition further comprising:
(a) cyclohexanol, 4-isopropyl: propionate, cis;
(b) cyclohexanemethanol, alpha-methyl: acetate;
(c) butyric acid: 1-cyclohexylethyl ester; and
(d) cyclohexanemethanol, alpha-methyl,

wherein the ratio of (a):(b):(c):(d) is about 3.8:1.5:0.4:0.5, and wherein zinc ricinoleate is at a level of about 2% by weight.

In another further embodiment, the malodor reducing composition additionally comprises a solvent.

In another further embodiment, a malodor reducing garbage bag system contains about 1-5% by weight of the malodor reducing composition of the present invention.

In another further embodiment, a substantially fragrance free odor blocking garbage bag system comprising, in combination, a garbage bag including an impermeable, flexible inelastic sheet constructed from a polyethylene material and including a side wall with a closed bottom and an open top; and the malodor reducing composition of the present invention impregnated within the sheet of the garbage bag.

The following examples are not meant to define or otherwise limit the scope of the invention. Rather the scope of the invention is to be ascertained according to the claims that follow the examples. Unless noted to the contrary, all percentages are given on a weight percent on a dry basis. All U.S. patents and published applications are hereby incorporated by reference as if set forth in their entirety.

### EXAMPLE I

### Malodor Reducing Garbage Bag System

Malodor: 3% IFF synthetic Garbage Malodor in Diethyl Phthalate (DEP)

Bag Lining Preparation:
Garbage bag (double film layer) was cut with scissors into two portions; a rectangle of 2.5×4.5 inch (L×W) dimension and a circle of 2.2 inch diameter (see figure). Note: Garbage bag number varies with test so it is indicated with the result table.
The rectangular portion was cut such that the closed edge of the garbage bag (which was taken from the side of the bag, 12″ from the bottom) is 2.5 inches and the longer side is 4.5 inches. The other 2.5 inch edge of rectangular portion was sealed using tape making the rectangle into a cylinder of 4.5 inch diameter. This cylindrical tube was placed inside the 8 oz jar to provide single bag layer interior lining.
The 2.2 inch circle (which is a single layer taken from 1″ below the rectangle piece) was placed inside the jar to cover the bottom of the jar.
A glass rod or spatula was used to adjust the lining such that it covers the sides and bottom of the jar completely. The jar was labeled with 3-digit blinding code for evaluation.
Under a fume hood, the lined jar was placed on weighing scale and 0.12 g of synthetic garbage malodor was added such that the malodor fell on the bottom and did not spray on the sides of jar. The lid was securely placed and the sample jars were not opened again until the evaluation.
All samples jars were prepared 17-19 hours prior to evaluation.

Sample for Analytical:
The remainder of the bag that was used for sensory testing is folded and placed in a foil bag.
Each sample was sealed in foil bag immediately after use.
The samples were submitted to analytical by late afternoon on the day before the test.

Subjects: 17-21 sensory panelists (female) trained on malodor intensity evaluation

Test Procedure:
Panelists follow standard procedure for sniffing jars. The jars were sniffed from a distance of about 3 inches from the top of the jar for 3 seconds by taking short sniffs.
Prior to the test panelists smell the synthetic garbage malodor presented in a jar lined with untreated film for familiarization.
The sample jars are presented in pairs. Each sample jar evaluated for malodor intensity using labeled magnitude scale (LMS) according to the procedure disclosed in Barry G. Green, Gregory S. Shaffer and Magdalena M. Gilmore, Derivation and Evaluation of a Semantic Scale of Oral Sensation Magnitude with Apparent Ratio Properties, Chemical Senses Vol. 18 no. 6 pp. 683-702, 1993.
Panelists enter their rating on tablet computers using Compusense® data collection software
Sample evaluation order is randomized between and within sample pairs

Analysis:
ANOVA to compare malodor intensities of samples within a pair. Significance established at p≦0.05.

| Sample | Malodor Intensity (Mean) | Std. Error of Mean |
|---|---|---|
| Control Sample | 11.86 | 1.14 |
| Technology Sample | 7.15 | 1.18 |

The garbage bag formulation comprises a combination of linear low density polyethylene and an acrylic styrene methyl methacrylate copolymer and a malodor reducing composition.

50%—Plastic LLDPE (Linear Low Density Polyethylene)

30%—Plastic Elvaloy® commercially available from Dupont

20%—Malodor reducing composition loaded in polyethylene material

The Control Sample contains Neobee M5 only. The Technology Sample contains the following:

| Ingredient | Weight (g) |
|---|---|
| Zinc Ricinoleate | 100 |
| Neobee M5 | 588.5 |
| Cyclohexanol, 4-isopropyl: propionate, cis (Veilex No. 4) | 189.322 |
| Cyclohexanemethanol, alpha-methyl: acetate (Veilex No. 2) | 76.650 |
| Butyric acid: 1-cyclohexylethyl ester (Veilex No. 1) | 18.933 |
| Cyclohexanemethanol, alpha-methyl (Veilex No. 3) | 26.595 |
| Total | 1000 |

It has been demonstrated in the above that the zinc ricinoleate-containing malodor reducing composition of the present invention (i.e., Technology Sample) is superior to the Control Sample in counteracting malodor.

### Example II

Preparation of Veilex Compositions: A series of Veilex compositions in DEP were prepared:

| Sample | Veilex Composition |
|---|---|
| 1 | Veilex No. 4 (2.8%) |
| | Veilex No. 2 (0.5%) |
| 2 | Veilex No. 4 (4.8%) |
| | Veilex No. 2 (2.5%) |
| 3 | Veilex No. 4 (2.8%) |
| | Veilex No. 2 (0.5%) |
| | Veilex No. 1 (0.1%) |
| | Veilex No. 3 (0.25%) |
| 4 | Veilex No. 4 (4.8%) |
| | Veilex No. 2 (2.5%) |
| | Veilex No. 1 (1%) |
| | Veilex No. 3 (1%) |
| 5 | Veilex No. 4 (2.8%) |
| | Veilex No. 2 (0.5%) |
| | Veilex No. 1 (0.1%) |
| 6 | Veilex No. 4 (4.8%) |
| | Veilex No. 2 (2.5%) |
| | Veilex No. 1 (1%) |

Establishment of A Malodor Model: A sweat model was prepared based on IFF's proprietary formulation.

Preparation of Test Samples: 0.5 grams of a Veilex sample and 0.1 grams of the malodor material as prepared above were pipetted into separate aluminum weighing dishes located at the bottom of a wide-mouth glass jar (240 mL). The jar was then capped and the samples were allowed to equilibrate for 24 hours before testing.

Testing Procedure: Sample pairs (Samples 1&3; 3&5; 2&4; and 4&6) were presented in a blind and pseudorandom order to a group of 20 trained panelists, who then entered a rating of malodor intensity using the Labeled Magnitude Scale (LMS). Mean (“Malodor Intensity”) and standard error of the mean (“SE”, ±) were obtained, where 0 represents “No Sensation” and 100 represents “The Strongest Imaginable Malodor Sensation”. ANOVA test was used to compare the rated intensities for each sample pair. Significance was at 95% Confidence Interval (p<0.05).

Results: The test results were as follows:

| Group | Sample Pair | Malodor Intensity | SE | P Value |
|---|---|---|---|---|
| 1 | Sample 1 | 12.63 | 1.08 | 0.03 |
| | Sample 3 | 9.47 | 1.15 | |
| 2 | Sample 3 | 10.47 | 1.12 | 0.34 |
| | Sample 5 | 11.46 | 1.10 | |
| 3 | Sample 2 | 7.39 | 1.14 | <0.01 |
| | Sample 4 | 5.02 | 1.17 | |
| 4 | Sample 4 | 4.51 | 1.16 | <0.01 |
| | Sample 6 | 8.03 | 1.13 | |

Specifically, compositions containing Veilex No. 1-4 of the present invention (Samples 3 and 4) were compared with compositions of Veilex No. 2 and No. 4 (Samples 1 and 2) and further with compositions of Veilex No. 1, No. 2, and No. 4 (Samples 5 and 6).

It has been shown clearly in the above that the Veilex compositions of the present invention (represented by Samples 3 and 4) are significantly superior to the other. Contrary to the understandings and expectations of the prior art (See, for example, U.S. Pat. No. 6,432,891), the inclusion of Veilex No. 3 in a Veilex Nos. 2 and 4-containing composition increased performance significantly.

### Example III

Preparation of Veilex Compositions: Additional Veilex compositions in DEP (Samples 7 and 8) were prepared:

| Sample | Veilex Composition |
|---|---|
| 4 | Veilex No. 4 (4.8%) |
| | Veilex No. 2 (2.5%) |
| | Veilex No. 1 (1.0%) |
| | Veilex No. 3 (1.0%) |
| 7 | Veilex No. 4 (4.8%) |
| | Veilex No. 2 (2.5%) |
| | Veilex No. 1 (0.05%) |
| | Veilex No. 3 (0.05%) |
| 8 | Veilex No. 4 (4.8%) |
| | Veilex No. 2 (2.5%) |
| | Veilex No. 1 (1.95%) |
| | Veilex No. 3 (0.05%) |

As described above in Example II, a malodor model was established and test samples were prepared. The test results were as follows:

| Group | Sample Pair | Malodor Intensity | SE | P Value |
|---|---|---|---|---|
| 5 | Sample 4 | 3.75 | 1.17 | 0.01 |
| | Sample 7 | 5.87 | 1.17 | |
| 6 | Sample 4 | 5.08 | 1.18 | 0.01 |
| | Sample 8 | 7.89 | 1.15 | |

It has been shown in the above that there is a criticality in the ratios of Veilex components in the compositions. A change of Veilex ratios caused significant loss in the effectiveness of compositions in counteracting malodor. Further, such loss could not be restored by randomly increasing the amount of a Veilex compound.

What is claimed is:

1. A zinc ricinoleate-containing malodor reducing composition further comprising:
(a) cyclohexanol, 4-isopropyl: propionate, cis;
(b) cyclohexanemethanol, alpha-methyl: acetate;
(c) butyric acid: 1-cyclohexylethyl ester; and
(d) cyclohexanemethanol, alpha-methyl,
wherein the ratio of (a):(b):(c):(d) is about 2.8-4.8:0.5-2.5:0.1-1:0.25-1, and wherein zinc ricinoleate is at a level of about 1-3% by weight.

2. The zinc ricinoleate-containing malodor reducing composition of claim 1, wherein the ratio of (a):(b):(c):(d) is about 3.8:1.5:0.4:0.5, and wherein zinc ricinoleate is at a level of about 2% by weight.

3. The zinc ricinoleate-containing malodor reducing composition of claim 1 further comprising a solvent.

4. A malodor reducing garbage bag system comprising, in combination,
(a) a garbage bag including a side wall with a closed bottom and an open top of an impermeable, flexible inelastic sheet constructed from a polyethylene material; and
(b) the zinc ricinoleate-containing malodor reducing composition of claim 1 impregnated within the sheet of the garbage bag.

5. The malodor reducing garbage bag system of claim 4, wherein the ratio of (a):(b):(c):(d) in the zinc ricinoleate-containing malodor reducing composition is about 3.8:1.5:0.4:0.5, and wherein zinc ricinoleate is at a level of about 2% by weight.

6. The malodor reducing garbage bag system of claim 4, wherein the zinc ricinoleate-containing malodor reducing composition is provided at a level of from about 1 to about 5% by weight of the malodor reducing garbage bag system.

7. The malodor reducing garbage bag system of claim 6, wherein the zinc ricinoleate-containing malodor reducing composition is provided at a level of about 2.5% by weight of the malodor reducing garbage bag system.
